(19) United States
(12) Patent Application Publication
(10) Pub. No.: US 2007/0162042 A1
(43) Pub. Date: Jul. 12, 2007

(54) Injection pump

(76) Inventors: Thomas Dunker, Teltow (DE); Dirk Hornscheidt, Berlin (DE); Frank Kniep, Grossbeeren/ Ot Kleinbeeren (DE)

Correspondence Address: WILLIAM COLLARD;COLLARD & ROE, P.C., 1077 NORTHERN BOULEVARD, ROSLYN, NY, 11576, US

(21) Appl. No.: 10/579,877

(22) PCT Filed: Nov. 18, 2003
(86) PCT No.: PCT/EP03/12888

§ 371 Date: May 18, 2006

(51) Int. Cl. A61F 2/00

(52) U.S. Cl. 606/94

(57) ABSTRACT

The invention relates to an injection pump for application of highly viscous media that have to be applied with high pressure, in particular during percutaneous vertebroplasty. Vertebroplasty relates to a method for treating bone pain which can occur, in particular, in spinal diseases or in other bones. Osteoporosis is the beginning of the disease, subsequently leading to tumoural diseases. The pain, in this group of diseases, is related to an increase of the loss of bone mass, which can also be associated with an increase in bone deformity. This deformity causes pain to the patient, the pain being a piercing, sometimes deep, drilling-like pain.

The invention relates to an injection pump for application of highly viscous media that have to be applied with high pressure, in particular during percutaneous vertebroplasty.

Vertebroplasty relates to a method for treating bone pain which can occur, in particular, in spinal diseases or in other bones. Osteoporosis is the beginning of said disease, subsequently leading to tumoural diseases. The pain, in said group of diseases, is related to an increase if the loss of bone mass, which can also be associated with an increase in bone deformity. Said deformity causes pain to the patient, said pain being a piercing, sometimes deep, drilling-like pain.

In vertebroplasty (or osteoplasty) the loss in bone mass is compensated by initial injection of viscid bone cement. Percutaneous Vertebroplasty is an effective new interventional method to treat bone pain. This therapeutic method has is already successfully employed in France and the US is capable of attaining a stabilisation of affected bones and a noticeable reduction of pain. Use of bone cement has become an established practice in endoprothetics. Long-term studies have shown that, with a stable implant position, spongy bone may very well remain vital in a cement embedding. Even grouting of vertebral bodies with bone cement has frequently proven successful in percutaneous interventional methods, with a focus on pain reduction. The respective bone is punctured by means of an application set under monitoring by roentgenology and/or computer tomography and with local anaesthesia, mostly in combination with neuroleptanalgesia. The needle is placed in the area of the pathologic fracture or bone tumour, respectively, and low-viscous bone cement is injected into the bone under permanent x-raying. The cement hardens after a brief period and provides new stability to the bone. During the intervention the patient is monitored in terms of blood pressure, oxygen saturation and pain symptoms.

On principle, it can be assumed that there are several opportunities available to inject the said bone cement after the above-described therapeutic method. Prior art knows several application devices to introduce bone cement in the described therapeutic method. Such devices for application of bone cement are described as having an enclosure that accommodates a cylinder to receive bone cement and a piston that can be moved in longitudinal direction and is arranged in the cylinder through which bone cement can be forced out through an outlet opening provided in the cylinder, with the piston for application of bone cement being movable in longitudinal direction under high pressure with a screw movement in the cylinder. A respective apparatus at the attached cannula in the bone to be treated is connected to the application device through a known LuerLock connection.

A number of requirements have to be met during application. On the one hand, filling of the application device and application into the affected bone structures have to be performed rather swiftly in a matter of a few minutes since the usually employed bone cements start to harden after 6 to 7 minutes after mixing. On the other hand, high-viscosity bone cement has to be applied under very high pressure since otherwise sufficient penetrance of bone structures is not ensured. Ultimately, application of bone cement must be well controllable since, in particular during application in the area of the spine, misrouting of bone cement may entail irreversible damage, such as vascular obstruction and ensuing embolism.

Furthermore known is patent specification DE 100 64 202 “Apparatus for the application of bone cement and a cannula for such an apparatus”, wherein an application device is described that facilitates filling of the cylinder by a lifting movement of the piston, i.e. by a direct displacement of the piston in longitudinal direction, in a very brief period. Conversely, liquid bone cement contained in the cylinder can then be applied by direct displacement of the piston in a short time until the generated counter-pressure becomes so high that it can no longer be overcome by the direct advance movement. AT that moment, the application device is switched to another mode—displacement of the piston by screw movement—since a screw movement allows for an essentially higher pressure to be exerted on the piston and thus on the bone cement to be applied than with a direct advance movement.

Especially the applicability of a bolted connection, that is known in different application devices for bone cement, makes the fundamental disadvantage of prior art. Especially exercise of the screw movement prevents direct connection between transmitting power during application of bone cement by the treating physician till of bone cement escapes. Due to the power transmission via threads or other force-influencing gears, the applying physician is not capable of controlling the direct interconnection between bone cement and pressure load.

There is a further disadvantage of the application device as described in the solution set forth in DE 100 64 202, therein that an additional extension has to be screwed to the application device to prevent that the treating physician is exposed to radiation during monitoring by roentgenology and/or computer tomography since he gets into the radiation area when applying bone cement with the described apparatus.

The task of the present invention to specify an injection pump for application of highly viscous media, that have to be applied with high pressure, in particular for employment in percutaneous vertebroplasty, with the application having to be performed in a brief period of time while simultaneously the necessary high pressure can be built up and monitoring by roentgenology and/or computer tomography can be provided without exposing the treating physician to radiation.

According to the invention the task is solved based on an injection pump of the type mentioned above in such manner that the injection pump for application of highly viscous media, that have to be applied with high pressure, in particular for employment in percutaneous vertebroplasty, is executed in compliance with Claim 1 and its Sub-claims. Thus, an injection pump is created that operates after a well-known pumping principle. A long pump body is provided with a low area volume in order to attain a low effort during pressing out the highly viscous medium from the distal opening of injection pump.

Within the framework of this application, the term “proximal” is used in the meaning of “disposed toward the body of the physician”. The term “distal” is used accordingly to mean “disposed remote from the body of the physician”.

The injection pump is fitted with a piston rod grip at the proximal end of the pump that has a rigid piston rod stretching through the grip of injection pump into the pump body. A flexible piston rod is mounted to this rigid piston rod which adapts itself to the deformed or flexible, as the case may be, pump body. The pump body is designed in such manner that it is either provided in a rigid deformation or can be employed according to a given application flexible and deformable by use of plastic material. The length of the rigid piston rod with the attached flexible piston rod ha been chosen so that at the end of the flexible piston rod a piston head is flush with the distal end within the pump body. Moreover, the distal end of the pump body is fitted with a hose bracket sleeve and a rotatable male LuerLock that serve to connect a cannula or needle used by the physician. During taking up of the highly viscous medium, e.g. bone cement, with the injection pump a nozzle is screwed into this rotatable male LuerLock that can be removed after filling the injection pump with a highly viscous medium and thus ensure a clean connection to a respective cannula. The length of the pump body is designed so that the performing physician can smoothly and with low effort inject highly viscous medium through the connected cannula via the piston rod grip after the injection pump has been filled and thus has an accurate feeling during injecting highly viscous medium into the affected bone by direct advance movement via the piston rod grip of the rigid piston rod and the connected flexible piston rod till to the piston head. This constitutes an essential advantage of the described injection pump, since there is a direct connection between effort applied and emergence of highly viscous medium at the distal end of the injection pump. The treating physician has at any rate the opportunity to determine guidance of the device by his own effort and the resulting emergence of highly viscous medium at the distal end of injection pump and thus has a better feeling of being in control of the emergence of the highly viscous medium. There is also the opportunity of preventing excessive emergence of highly viscous medium into damaged vertebra parts by slightly pulling back the piston rod grip during injection of highly viscous medium and thus relieve pressure in the pump system which allows for a correct placing of highly viscous medium in the damaged bone. A technical solution is also provided for the piston head located at the distal end which has a valve effect wherein air that is present in the pump body is forced out when highly viscous medium pressed in. The piston head is designed in such a manner that it has a centre boring whose rear part if filled with a filter, e.g. Cellulose or foam material that is air permeable. An overpressure is generated when the highly viscous medium contained in the pump body is pressed out by the effort applied by the treating physician, which overpressure is discharged via the filter and a vertical boring in the piston head. A valve hose is arranged above this vertical boring and provides for a valve effect for air to escape when highly viscous medium ism pressed in.

Below the invention is further explained in greater detail and by means of four drawings.

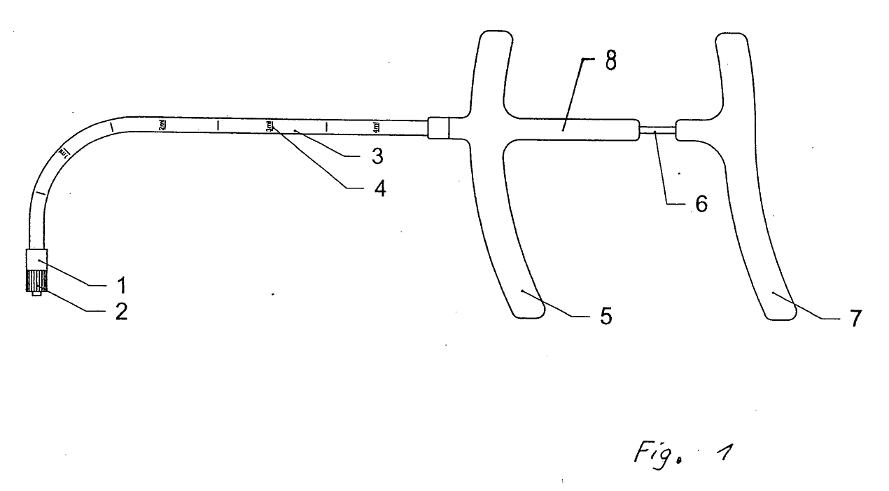

FIG. 1 shows the injection pump;

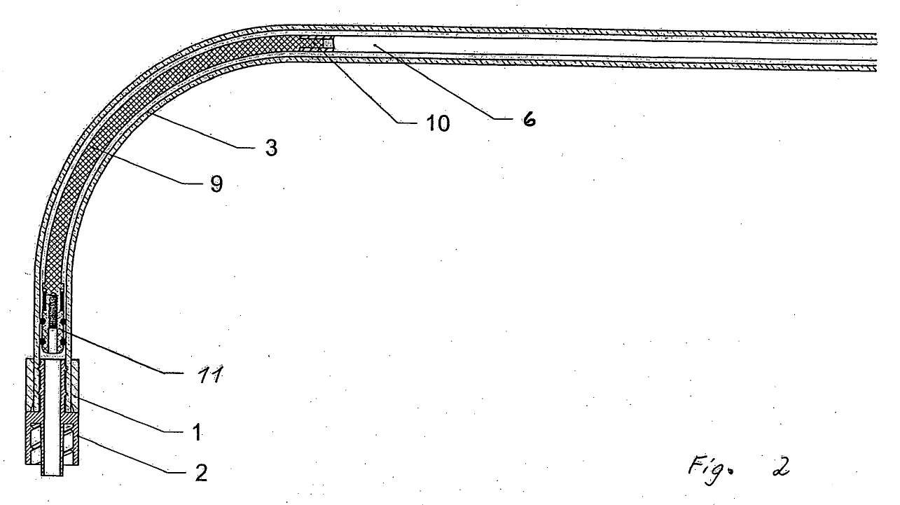

FIG. 2 shows the design of the pump body;

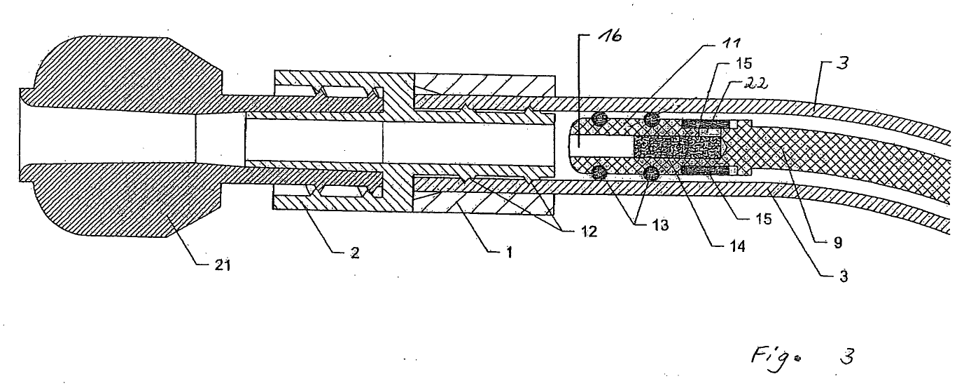

FIG. 3 shows the distal end of the pump body;

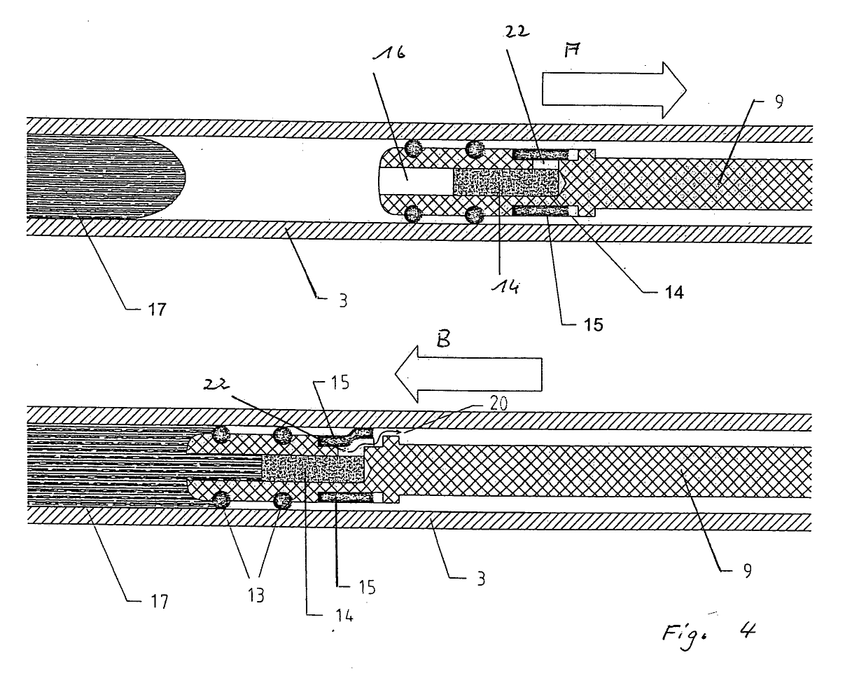

FIG. 4 shows suction and pressing out of bone cement inside the pump body.

FIG. 1 shows an injection pump 8 in a normal view, wherein the injection pump 8 is composed of a piston rod grip 7 that fastened to the distal end of the rigid piston rod 6 which is arranged so that it can displaced through a grip 5 of the injection pump 8 into the pump body 3. The pump body 3 is specified in such a manner that it is either preformed as a rigid body or can be flexibly deformable by attaching a plastic hose to the pump body 3. The pump body 3 is provided with ml markings 4 to indicate the bone cement content. A hose bracket sleeve 1 with a rotatable male LuerLock 2 is arranged at the distal end of the pump body 3. This rotatable male LuerLock 2 serves the connection of an employed cannula or needle that is previously placed by the physician with suitable aids into the bone to be treated. The embodiment of the invention according to FIG. 1 shows the depiction of the injection pump 8, wherein grip pieces 5 and 7 are shaped in such a manner that easily handling of the respective injection pump 8 is ensured. The graphic presentation in FIG. 1 shows the injection pump 8 in an initial state in which no bone cement 17 has been sucked yet into the pump body 3. Hence, it can be stated that particular the rigid piston rod 6 projects in closed condition with t certain section from the grip 5. On principle, it can be assumed that especially this rigid piston rod 6 projects towards the distal end at any rate, e.g. by not less than one centimetre, into the pump body 3 in order to provide adequate stability. The rigid piston rod 6 is preferably made of metal. The connection between pump body 3 and grip 5 can be executed fixed, rotatable and replaceable. Manufacturing the pump body 3 of plastics makes it flexibly deformable and, depending on a given application, bendable when filled with bone cement so that it can be smoothly airtight connected to a cannula placed above the rotatable male LuerLock 2. The pump body 3 can be executed in a variety of variants, e.g. that the body is manufactured in a pre-shaped condition with a flexible piston rod 9 being attached to the rigid piston rod 6 at the distal end that adjusts itself in any case to the deformity of the pump body 3. The grip 5 has a vertical length of ca. 10 cm. In addition, the pump body 3 is attached to the grip 5 with a length of ca. 22 cm. These dimensions can be relatively modified according to a given application, in that the pump body 3 may be executed shorter or longer. Also different grip forms of the piston rod grip 7 can be chosen. As already described in the invention, the rigid piston rod 6 projects from the side of the piston rod grip 7 beyond the grip 5 into the pump body.3, whereby two thirds of the overall length of the injection pump 8 appear to be advantageous. The remaining dimension is provided with the flexible piston rod 9 which adapts to the pre-formed shapes or a pump body 3 of plastics.

FIG. 2 shows the interior configuration of the pump body 3. It can be seen that the rigid piston rod 6 ends before any bend and subsequently a flexible piston rod 9 is arranged via a respective connection of the two piston rods 10 at this rigid piston rod 6. The flexible piston rod 9 is preferably made of plastics but can also be of spring steel or other flexible, solid materials which allow for adaptation to the pre-formed or flexible pump body. A piston head 11 in pushed together state is arranged at the distal end of the flexible piston rod 9 which ends immediately before the hose bracket sleeve 1 with downstream rotatable male LuerLock 2. This piston head 11 is fitted with sealing rings 13 sand provides for a suction effect upon taking up of bone cement. Preferably the pump body 3 in a special embodiment has a length of ca. 20 to 25 cm and is made of plastics, whereby a flexibly formed pump body 3 can be connected by the physician to an inserted cannula via the distally arranged rotatable male LuerLock 2.

FIG. 3 shows the distal end of the injection pump 8, the detailed configuration of the piston head 11 at the distal end of the flexible piston rod 9, and the arrangement of the hose bracket sleeve 1 with attached rotatable male LuerLock 2 and a nozzle 21 that is screwed into the rotatable male LuerLock 2 and serves as nozzle to draw in bone cement 17 from a tank. After the injection pump 8 has been filled with the specified quantity of bone cement 17 the nozzle 21 is unscrewed from the rotatable male LuerLock 2 to ensure that the rotatable male LuerLock 2 can be cleanly placed onto a LuerLock connection. What is important in this context is that the connections between pump body 3, hose bracket sleeve 1 and the contained rotatable male LuerLock 2 are airtight so that there is no air ingested during drawing in or pressing out, of bone cement 17.

In this special embodiment of the technical solution the male LuerLock 2 is rotatable and arranged to ensure tightness by fixing the hose bracket or pump body 3 in the male LuerLock 2 by fitting the LuerLock 2 with prongs 12 into which the pump body 3 is radially forced to fasten the hose bracket sleeve 1.

Any air ingress into the pump body 3 during ingestion of bone cement 17 is discharged by a specially fitted vent at the piston head 11. The piston head 11 is arranged at the distal end of the flexible piston rod 9. Double sealing rings 13 are arranged at defined distances piston head 11 and the internal wall of the pump body 3 in order to ensure the suction effect during intake of bone cement 17. The distance has been deliberately chosen to maintain airtightness even when pump body 3 is bent. A venting boring 16 is provided at the centre of the piston head 11. This boring is executed in such a manner that it projects two thirds of the length from the distal end into the piston head 11. The boring 16 is lined with cellulose 14 up to half its height. This cellulose 14 has such properties that it becomes air permeable at respective pressure conditions, i.e. a slight overpressure of ca. 0.01 bar. Yet, also other materials, such as foam or air permeable materials that act as filters are conceivable. At the end of centre boring 16 there is a vertical boring 22 provided that is connected with the centre boring 16. A valve hose 15 is arranged radially above this vertical boring 22, it is executed flexibly, in particular for certain pressure conditions, on top of the boring 22. This valve hose 15 serves in particular for venting drawn in bone cement 17, as described below for FIG. 4.

Another embodiment provides for the two sealing rings 13 and the valve hose 15 being executed in a special type of construction in such a manner that a single sealing is arranged so that also a valve hose effect is achieved. Thus, a sealing sleeve is provided which simultaneously creates a valve effect for venting.

FIG. 4 shows a moving direction “Suction A” and a moving direction “Pressing out B” of bone cement 17. Moving direction “Suction A” shows that bone cement 17 is drawn in from a tank by pulling out the flexible piston rod 9 from the pump body 3 with subsequent rigid piston rod 6 via the piston rod grip 7. A shown in the drawing, a certain air cushion is created between bone cement 17 and distal end of the flexible piston rod 9 till to the piston head 11, depending on how the injection pump 8 is handled. The generated air bubbles and air cushions have to be removed from the pump body 3 to prevent any air ingress during injection of bone cement 17 into the respective bone of the patient's bone segment through an inserted cannula. Moving direction “Pressing out B” indicates that the flexible piston rod 9 with piston head 11 is moved in distal direction till to the bone cement 17. An air outlet 20 can now be provided through the cellulose 14, vertical boring 22 and the opening valve hose 15 due to the generated overpressure and the tight connection via the sealing rings 13 till into the centre boring 16 of the cellulose 14. Thus, sliding of piston rods 9 and 6 towards the distal end makes sure that the contained bone cement 17, previously drawn in, is vented. Drawing 4 shows that disturbing air is ingested during filling of the injection pump 8 because the consistency of the material is mostly very viscid, this is why it is recommended to perform a venting operation by using the valve effect as described above for the piston head 11.

Below is a description for handling the practical example of the injection pump 8 as described therein and the pertaining advantages. Vertebroplasty is a new method for percutaneous augmentation of vertebral bodies with bone cement. This technique is employed to stabilise a weakened fractured vertebral body and decisively improve the pain symptoms in a patient. In face-down position and under radioscopy with CT or MRT methods the vertebral body is punctured across the pedicle with a bone puncture needle. Additionally, a freshly mixed, sterile and liquid bone cement (PMMA—polymethyl methacrylate) is injected. This cement basically resembles the material that has been used for decades to cement in joint prostheses. Subsequently, bone cement 17 is sucked up via the injection pump 8 with nozzle 21 at the distal end to the hose bracket sleeve 1 in combination with the rotatable male LuerLock 2. The nozzle 21 is already screwed into the LuerLock 2 for this process. After the specified quantity of bone cement 17 has been drawn into the injection pump 8 the nozzle 21 is unscrewed at the distal end of injection pump 8 from the LuerLock 2. Ingested air can be filtered out via the venting opening at the piston head 11 by slightly pressing the pump towards the distal end. Then the injection pump 8 is placed onto the bone puncture needle via the LuerLock connection. The following injection of bone cement 17 through the injection pump 8 is also made as shown in the figure so that the entire cement injection is well controllable. The essential advantages of handling the injection pump 8 are that it allows for a flexible arrangement by the flexible pump body 3 or a pre-formed pump body 3 because in particular in certain imaging method where there is only confined space to attach a respective injection pump 8 to an inserted needle. It shall be particularly mentioned here that the CT method only provides an area of only ca. 10 to 30 mm to place the injection pump 8. Moreover, the size of the injection pump 8 can prevent the treating physician from getting into the radiation sphere of the imaging method. A very good handling during injection of bone cement 17 through the needle is achieved in the application of bone cement 17 contained in the injection pump 8 due to the positive power ratios, length of cannula and diameter of cannula. The invention gives the treating physician the opportunity to control the quantity of injected bone cement 17 via the pump effect through the direct contact with the power effect on the piston rod grip 7 during pressing in. Additionally, a pressure relief can be attained during injection of bone cement 17 by slightly pulling the piston rod grip 7 back. Another essential feature is the construction of the injection pump 8 and the flexible design of a pump body 3 the device can be easily attached to a respective needle through the LuerLock connections at both instruments. The specified length of the injection pump 8 allows its application in an imaging method without any problems.

### REFERENCE CHARACTERS

1 Hose bracket sleeve
2 rotatable male LuerLock
3 Pump body
4 ml marking on pump body
5 Grip for injection pump
6 rigid piston rod
7 Piston rod grip of injection pump
8 Injection pump
9 flexible piston rod
10 Connection rigid piston rod with flexible piston rod
11 Piston head
12 Prong
13 Sealing rings
14 Cellulose
15 Valve hose
16 Venting boring
17 Bone cement
20 Air outlet
21 Nozzle
22 vertical boring

1. Injection pump for application of highly viscous media that have to be applied with pressure, in particular during percutaneous vertebroplasty, in which a piston system with grip ends to take up bone cement is provided in a piston, wherein a piston rod (6) is rigidly arranged at a piston rod grip (7) of the Injection pump (8) and the distal end of the rigid piston rod (6) is provided with a flexible piston rod (9) to the distal end of a pump body (3) with an end piston head (11), where the pump body (3) is fastened at the proximal end at a grip (5) of the Injection pump (8).

2. Injection pump according to claim 1, wherein the length of the rigid piston rod (6) being dimensioned in such manner that the rigid piston rod (6) remains in the pump body (3) when the piston rod (6) is pulled out through the grip (5) by means with the piston rod grip (7).

3. Injection pump according to claim 1, wherein the pump body (3) is flexible or ductile with preferable use of a plastic material for the pump body (3).

4. Injection pump according to claim 1, wherein the pump body (3) has a rigidly bent shape.

5. Injection pump according to claim 3, wherein the flexible piston rod (9) is matched to the chose rigid deformation of the pump body (3).

6. Injection pump according to claim 3, wherein the flexible piston rod (9) is matched to the shape of the pre-formed pump body (3).

7. Injection pump according to claim 1, wherein the flexible piston rod (9) is fitted at its end with a relatively soft or flexible material, preferably a plastic material.

8. Injection pump according to claim 1, wherein a piston head (11) is arranged in the pump body (3) at the distal end of the flexible piston rod (9), with sealing rings (13) between piston head (11) and pump body (3) to create a suction effect when pulling out the piston rods (6 and 9) in proximal direction.

9. Injection pump according to claim 1, wherein a hose bracket sleeve (1) with an attached rotatable male LuerLock (2) at the distal end of the pump body (3).

10. Injection pump according to claim 9, wherein a nozzle (21) is screwed to the rotatable male LuerLock (2) to take up highly viscous media from a respective vessel which nozzle (21) can be unscrewed after absorption of such highly viscous media.

11. Injection pump according to claim 8, wherein the piston head (11) at the flexible piston rod (9) has a centred venting boring (16), with the rear section of the boring (16) being equipped with an air-permeable filter, preferably of foam material or cellulose (14).

12. Injection pump according to claim 8, wherein the proximal end of the centred venting boring (16) in the piston head (11) is provided with a vertical boring (22), which vertical boring (22) is radially covered with a valve hose (15).

13. Injection pump according to claim 9, wherein the male LuerLock (2) is fitted with a prong (12) to fasten the pump body (3) by radially pressure-forcing the pump body (3) into place.

14. Injection pump according to claim 1, wherein the pump body (3) is arranged at the grip (5) firmly, rotatable and replaceable.
